(19) United States
(12) Patent Application Publication
(10) Pub. No.: US 2010/0041115 A1
(43) Pub. Date: Feb. 18, 2010

(54) PRODUCTION OF DICARBOXYLIC ACIDS BY IMPROVED MUTANT STRAINS OF YARROWIA LIPOLYTICA

(76) Inventors: Jean-Marc Nicaud, Trappes (FR); France Thevenieau, Paris (FR); Marie-Thérèse Le Dall, Genevilliers (FR); Rémy Marchal, Chatou (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/721,726

(22) PCT Filed: Dec. 13, 2005
(86) PCT No.: PCT/FR2005/003140

§ 371 Date: Jul. 6, 2009

(30) Foreign Application Priority Data
Dec. 15, 2004 (FR) 0413468

(51) Int. Cl. C12P 7/46; C12N 15/80; C12N 1/15

(52) U.S. Cl. 435/145; 435/471; 435/254.11

(57) ABSTRACT

The invention concerns a method for producing dicarboxylic acids (DCA) with long hydrocarbon chains, also called diacids, which consists in culturing a mutant strain of Yarrowia lipolytica obtained by mutagenesis directed and more particularly disrupted at least for the POX2, POX3, POX4 and POX5 genes encoding acyl-CoA oxydase, in a medium consisting essentially of an energetic substrate including at least one carbon source and one nitrogen source and in subjecting said strain to a bioconversion substrate selected among n-alkanes of at least 10 carbon atoms, fatty acids of at least 10 carbon atoms, their alkyl esters and natural oils.

### FIELD OF THE INVENTION

The invention relates to a method of producing dicarboxylic acids by fermentation using a mutant strain of the yeast Yarrowia lipolytica from a bioconversion substrate.

Dicarboxylic acids (also referred to as “diacids”) are used as base material for the synthesis of polyamides and polyesters, lubricating oils, plasticizers or perfumes.

Diacid production methods vary according to the number of carbon atoms of the carbon network of the diacid considered (Johnson R W, Pollock C M, Cantrell R R, Editors Kirk-Othmer Encyclopedia of Chemical Technology, 4^{th} Edition, 1983, pp. 118-136). Thus, azelaic acid (C9 diacid) is conventionally obtained by chemical oxidation of oleic acid through ozone whereas sebacic acid (C10 diacid) is produced by alkaline oxidation of ricinoleic acid. Dodecanedioic acid (C12 diacid) is a product of petrochemnistry. Microbiology is used to produce brassylic acid (C13 diacid) from tridecane.

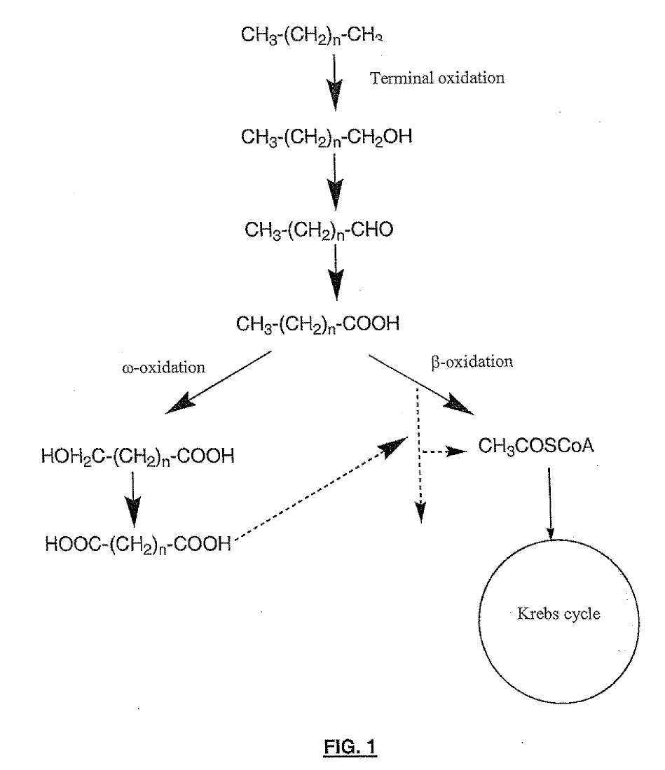

Considering the variety of diacids used in the various applications, the advantage of a production channel applicable to the widest range of diacids possible is unquestionable. Although characterized by a lower reaction rate than chemical production, biological production affords the advantage of being applicable to a great variety of substrates (the biological diacid production process is diagrammatically shown in FIG. 1).

In fact, many wild microbial species such as Cryptococcus neoformans, Pseudomonas aeruginosa, Candida cloacae, etc., are capable of excreting small amounts of diacids (Chan et al.: Stimulation of n-alkane conversion to dicarboxylic acid by organic-solvent- and detergent-treated microbes. Appl. Microbiol. Biotechnol., 34, 1991, 772-777; Shiio et al.: Microbial Production of Long-chain Dicarboxylic Acids from n-Alkanes. Part I. Screening and Properties of Microorganisms Producing Dicarboxylic Acids. Agr. Biol. Chem. 35, No. 13, 1971, p. 2033-2042).

However, in order to obtain substantial amounts of diacid excretions, mutants blocked in 1-oxidation have to be used. For many species, such mutants were obtained by random mutagenesis, followed by a suitable selection (patent EP 0 229 252 B1 Shiio et al.; Jiao et al. Isolation and Enzyme Determination of Candida tropicalis Mutants for DCA Production. J. Gen. Appl. Microbiol. 2000, 46: 245-249).

A much more elegant but more restricting option using directed mutagenesis techniques has however been developed for Candida tropicalis. From a wild strain belonging to this species, Picataggio et al. sequentially disrupted the four genes coding the two isoenzymes of acetyl-CoA oxidase (Aox) that catalyse the first β-oxidation stage (Determination of Candida tropicalis Acylcoenzyme A Oxidase Isoenzyme Function by Sequential Gene Disruption. Mol. Cell. Biol. 11, 1991, 4333-4339, and U.S. Pat. No. 5,254,466 A1).

These authors then overexpressed the genes coding for cytochrome P450 mono-oxygenase and NADPH-cytochrome reductase, that constituted the activities limiting the kinetics of the conversion of n-alkanes to diacids (Picataggio et al.: Metabolic Engineering of Candida tropicalis for the Production of Long-chain Dicarboxylic Acids. Biotechnol. 10, 1992, 894-898, and U.S. Pat. No. 5,648,247 A1). However, the Candida tropicalis mutants produced according to a multicopy amplification system do not seem to be totally stable and they possibly undergo reversions. This is the reason why improvements had to be carried out in the prior art (patent application US 2004/0014198 A1).

### OBJECT OF THE INVENTION

The object of the invention is to overcome the drawbacks of the prior art. It has in fact been discovered that it is advantageously possible to produce diacids using Yarrowia lipolytica mutants whose genes coding for acyl-CoA oxidase were disrupted.

The diacids that the method according to the invention aims to prepare are organic compounds with a linear hydrocarbon chain having at least 10 carbon atoms comprising a carboxylic function at each end of the chain.

The microorganism used is a Yarrowia lipolytica mutant wherein at least the POX2, POX3, POX4 and POX5 genes were disrupted so as to block it partly in β-oxidation.

Apart from the acyl-CoA oxidases coded by the POX2, POX3, POX4 and POX5 genes, two other acyl-CoA oxidases coded by the POX1 and POX6 genes, of unknown function, are present in the genome of Yarrowia lipolytica.

These two acyl-CoA oxidases are involved in the reconsumption of the diacids biosynthesized by sequential elimination of two carbon atoms. Additional disruption of the POX1 and POX6 genes thus leads to the production of diacids corresponding to the profile of the bioconversion substrate. For example, if oleic sunflower oil predominantly consisting of fatty acids with 18 carbon atoms is used as the bioconversion substrate, the strain deleted of all of the POX genes will produce a majority of diacids with 18 carbon atoms.

On the other hand, the bioconversion substrate can be stored in form of triglycerides within the cell in form of lipid bodies and thus become inaccessible for its bioconversion to diacids. Genes were identified, by proteomic analysis, as being involved in the accumulation of the bioconversion substrate. We identified the following genes: DGA1 (acyl-CoA:diacylglycerol acyltransferase) TGL1 (triacylglycerol lipase), G3P (glycerol-3-phosphate dehydrogenase), SCP2 (putative sterol carrier), LR01 (putative lecithine cholesterol acyltransferase), as well as IFP 621 IPF (unknown function), IPF 905 (unknown function), and IPF 2569 (NADH-ubiquinone reductase subunit).

Modification of the activity of these genes (by disruption or overexpression) thus leads to a decrease in the accumulation of the bioconversion substrate in form of lipid bodies within the Yarrowia lipolytica cell.

It can be noted that the genetic system of Yarrowia lipolytica is very different from that of Candida tropicalis. Unlike Candida tropicalis, which is a diploid yeast, Yarrowia lipolytica is in fact a haploid species. In the latter microorganisms, genic deletion operations are therefore much more efficient and surer because of the presence of a single set of chromosomes.

On the other hand, a promoter of the POX2 gene coding for acyl-CoA oxidase is used to overexpress genes such as, for example, those coding for P450 mono-oxygenase cytocbrome and for NADPH-cytochrome reductase. Promoter pPOX2 has the property of being highly inducible by bioconversion substrates. Thus, overexpression of a gene of interest is carried out by addition of a single gene copy under the control of promoter pPOX2, allowing to obtain efficient, stable and non-reverting Yarrowia lipolytica mutants, unlike the Candida tropicalis mutants obtained by means of a multicopy amplification system.

Another advantage of Yarrowia lipolytica over Candida tropicalis for the production of diacids from fatty acid esters or natural oils, vegetable oils for example, is as follows: the conversion of natural oils to diacids by Candida tropicalis requires at least partial chemical hydrolysis of the substrate prior to fermentation (see U.S. Pat. No. 5,962,285). This hydrolysis is carried out by saponification performed in the presence of calcium or magnesium hydroxide. It produces the corresponding fatty acid salts (soaps). Now, Yarrowia lipolytica has the capacity of assimilating triglycerides as the carbon source. The first stage of this catabolism involves hydrolysis of the triglycerides to free fatty acids and glycerol by the lipolytic enzymes (lipases) identified by Peters and Nelson in 1951. An extracellular lipase activity and two membrane lipases of 39 and 44 kDa (Barth et al., Yarrowia lipolytica in: Nonconventional Yeasts in Biotechnology A Handbook (Wolf, K., Ed.), Vol. 1, 1996, pp. 313-388. Springer-Verlag) were described thereafter. Yarrowia lipolytica can produce several lipases (extracellular, membrane and intracellular activity). Recently, the genes corresponding to the lipases described have been identified. The LIP2 gene codes for an extracellular lipase, Lip2p (Pignede et al., 2000). It has been shown that it preferably hydrolyzes the long-chain triglycerides of oleic residues (Barth et al., 1996).

Yarrowia lipotytica thus directly hydrolyzes esters and natural oils to free fatty acids and glycerol under pH conditions compatible with fermentation conducting. Under such conditions, hydrolysis of ester or of oil and its conversion to diacids occur simultaneously, which has the advantage of leading to a simplified operating protocol since the chemical hydrolysis stage is eliminated.

### DETAILED DESCRIPTION

In a more detailed manner, the invention provides a method of producing at least one dicarboxylic acid, comprising:

(a) a growth stage wherein a mutant strain of Yarrowia lipolytica disrupted at least for the POX2, POX3, POX4 and POX5 genes (coding for acyl-CoA oxidase) is cultured in a culture medium essentially consisting of an energetic substrate comprising at least a source of carbon and a source of nitrogen,

(b) a bioconversion stage wherein said strain is subjected to a bioconversion substrate selected from among the n-alkanes having at least 10 carbon atoms, fatty acids having at least 10 carbon atoms, alkyl esters having 1 to 4 carbon atoms of these fatty acids, such as mixtures of methyl or ethyl esters, or natural oils (mixtures of fatty acid esters of glycerol), in the presence of an energetic substrate, and

(c) a stage of recovering the dicarboxylic acid formed.

The strains used in the method according to the invention derive from the wild Yarrowia lipolytica W29 strain (ATCC 20460, recorded under CLIB89 in the Collection de Levures d'Interet Biotechnologique-CLIB).

It is thus possible to use new mutant strains derived from the Yarrowia lipolytica ATCC 20460 strain by means of the Pold strain [strain auxotrophic for leucine (leu−) and uracil (ura−)] described in G. Barth et al.'s review. It is recorded under CLIB139 in the CLIB.

The way these new mutant strains (MTLY37, MTLY66, MTLY74, MTLY79, MTLY80, MTLY81, FT 120 and FT 130) are obtained is described below.

In the diacid production method according to the invention, the mutant strain selected is cultured in a medium essentially consisting of an energetic substrate that comprises at least a source of carbon and a source of nitrogen until growth end. The bioconversion substrate (alkanes or alkane mixtures, fatty acid or fatty acid mixtures, fatty acid ester or fatty acid ester mixtures or natural oil or mixtures of these various substrates) is then added so as to initiate the bioconversion to diacids and the diacids formed are recovered by means of a technique known to the man skilled in the art, such as calcium salt precipitation.

During the bioconversion stage, the culture medium can involve a supply of secondary energetic substrate generally consisting of at least one polyhydroxyl compound such as, for example, glycerol or a sugar.

1. Obtaining the Mutant Strain MTLY37

This procedure for obtaining this strain can be as follows:

1) sequencing the gene of interest (or the sequence of this gene is available in a data bank),

2) constructing a disruption cassette by PCR (Polymerase Chain Reaction) or by cloning, using a counter-selectable marker, for example marker URA3 (with which one can select for the Ura+ phenotype or for the Ura− phenotype),

3) selecting the strains with the deleted gene of interest (conversion and selection of the transformers) (advantageously Ura+ if the marker is URA3) and checking the gene disruption.

A particular method of obtaining the mutant strain MTLY37 is described hereafter.

POX genes of the wild strain, whose sequences are different from those of Candida tropicalis, are first cloned and sequenced. Disruption cassettes for the genes coding for the iso-enzymes of acyl-CoA oxidase are then constructed. The genes of acyl-CoA oxidase are disrupted using the selectable marker URA3. The promoter and terminator zones are amplified by a first PCR, using specific oligonucleotide pairs, which eliminates the complete sequence from the open reading frame (ORF). A second PCR is then carried out with the external primers and the PCR products of the promoters and terminators, which merge via a common extension of 20 bp comprising a site for the restriction enzyme I-Scel. The PCR product is cloned to give a series of plasmids (designated by pPOX-PT) containing the promoter-terminator module (disruption cassette 2).

A URA3 gene is introduced into the I-Scel site of the POX-PT cassette. A series of pPOX-PUT plasmids containing the promoter-URA3-terminator module is constructed (disruption cassette 1). These constructions are referred to as pPOXI-PUT, pPOX2-PUT, pPOX3-PUT, pPOX4-PUT and pPOX5-PUT for the plasmids containing disruption cassette 1, on the one hand, and pPOX1-PT, pPOX2-PT, pPOX3-PT, pPOX4-PT and pPOX5-T for the plasmids containing disruption cassette 2, on the other hand.

The disruption cassettes are amplified by PCR with the specific external primers, using for example the Pfu polymerase (provided by Stratagene, La Jolla, Calif.). The final analysis of the protein sequences shows that the acyl-CoA oxidases of Yarrowia lipolytica have an identity degree of 45% (50% similarity) with that of the other yeasts. The identity degree between them ranges from 55 to 70% (65 to 76% similarity).

After constructing the disruption cassettes of the genes coding for acyl-CoA oxidase, the conversion of Yarrowia lipolytica can be carried out by means of various methods. Electroporation can be performed, wherein the DNA is introduced by means of the electric shock. More advantageously, the lithium acetate and polyethylene glycol method can be used. It is described by Gaillardin et al.: LEU2 Directed Expression of Beta-gallactosidase Activity and Phleomycin Resistance in Yarrowia lipolytica. Curr. Genet. 11, 1987, 369-375.

The presence of disruption is checked by PCR according to Gussow et al.'s technique: Direct Clone Characterization from Plaques and Colonies by the Polymerase Chain Reaction. Nucleic, Acids Res. 17, 1989, 4000, then confirmed by Southern blot hybridization.

At the start of the Po1d strain, disruptions are carried out in two stages.

Po1d is first converted with the PCR PUT disruption cassette 1 and selected. The Ura+ clones are then converted with disruption cassette 2 to eliminate the URA3 gene and they are selected. This protocol allows to obtain the disrupting quadruple MTLY37-pox2ΔPT-pox3ΔPT-pox4ΔPT-pox5ΔPUT. The diagrammatic representation of the construction of this mutant is summed up in Table I hereunder.

TABLE 1
Conversion stages required for the production of mutant MTLY37
no longer growing on oleic acid

| | Conversion operations | | |
|---|---|---|---|
| | | Conversion | |
| Stage | Mutant to be converted | cassette | Converted mutant |
| 1 | PO1d, Leu−, Ura− | POX5-PUT | MTLY15, Leu−, Ura+, |
| | | | Δ5-PUT |
| 2 | MTLY15, Leu−, Ura+, | LEU2 | MTLY19, Leu+, Ura+, |
| | Δ5-PUT | | Δ5-PUT |
| 3 | MTLY19, Leu+, Ura+, | POX5, PT | MTLY24, Leu+, Ura−, |
| | Δ5-PUT | | Δ5-PT |
| 4 | MTLY24, Leu+, Ura−, | POX2, PUT | MTLY29, Leu+, Ura+, |
| | Δ5-PT | | Δ5-PT, Δ2-PUT |
| 5 | MTLY29, Leu+, Ura+, | POX2-PT | MTLY32, Leu+, Ura−, |
| | Δ5-PT, Δ2-PUT | | Δ5-PT, Δ2-PT |
| 6 | MTLY32, Leu+, Ura−, | POX3-PUT | MTLY35, Leu+, Ura+, |
| | Δ5-PT, Δ2-PT | | Δ5-PT, Δ2-PT, Δ3-PUT |
| 7 | MTLY35, Leu+, Ura+, | POX3-PT | MTLY36, Leu+, Ura−, |
| | Δ5-PT, Δ2-PT, Δ3-PUT | | Δ5-PT, Δ2-PT, Δ3-PT |
| 8 | MTLY36, Leu+, Ura−, | MTLY18 | MTLY37, Leu+, Ura+, |
| | Δ5-PT, Δ2-PT, Δ3-PT | deleted for | Δ5-PT, Δ2-PT, Δ3-PT, |
| | | POX4 | Δ4-PUT |
| | | (Δ4-PUT) | |

Disruption of a gene and excision of the marker can also be done by means of a method involving a recombination or a recombinase. It is for example possible to use markers with, on either side, a repeated sequence (allowing the recombination that is selected) or a lox sequence that is recognized by the recombinase Cre. Excision occurs when the recombinase Cre is expressed, Fickers et al., 2003 New Disruption Cassettes for Rapid Gene Disruption and Marker Rescue in the Yeast Yarrowia lipolytica. J. Microbiol. Methods 55/3:727-737.

2. Obtaining the Mutant Yarrowia Lipolytica Strain MTLY74

The strain MTLY74 Leu+ Ura− is constructed from the mutant MTLY37.

2a. From the prototrophic mutant MTLY37 (Leu+, Ura+), we construct a mutant Yarrowia lipolytica strain MTLY66 auxotrophic for leucine and uracil (Leu−, Ura−). The first stage is the construction of the strain MTLY40 auxotrophic for uracil (Leu+, Ura−) by conversion of marker URA3 by marker ura3-41 by transforming the PCR fragment containing this marker and by selecting the Ura− in the presence of 5FOA. From the mutant MTLY40, we construct a mutant Yarrowia lipolytica strain MTLY64 auxotrophic for leucine (Leu−, Ura−, Hyg+) by disruption of marker LEU2 by converting the disruption cassette PHTleu2 and by selecting the resistant hygromycin transformers (leu2::Hyg). From the mutant MTLY64, we construct a mutant Yarrowia lipolytica strain MTLY66 auxotrophic for leucine (Len−, Ura−) by excision of the HYG marker by transforming the replicative vector pRRQ2 containing the recombinase Cre and marker LEU2 (Cre-LEU2) and by selecting the sensitive hygromycin transformers, Leu+. The loss of plasmid pRRQ2 is achieved by culture on a rich medium YPD and by isolation of a clone (Leu−, Ura−, Hyg−).

2b. From the mutant MTLY66, we construct a mutant Yarrowia lipolytica strain MTLY74 Leu+ Ura− that overexpresses the NADPH-cytochrome reductase, by expressing it under control of the strong promoter pPOX2, induced by the bioconversion substrates, of fatty acid, fatty acid ester or natural oil type.

The gene coding for NADPH-cytochrome reductase (CPR) is introduced in a vector containing the selection gene LEU2, JMP21 for example, under the control of promoter pPOX2 inducible by the fatty acids, fatty acid esters or natural oils. The marker-promoter-gene cassette (LEU2-pPOX2-CPR) is introduced by conversion.

The diagrammatic representation of the construction of this mutant is summed up in Table 2 hereunder.

TABLE 2
Conversion stages required for the production of mutant MTLY74 from
MTLY37

| | Conversion operations | | |
|---|---|---|---|
| Stage | Mutant to be converted | Conversion with | Converted mutant |
| 1 | MTLY37, Leu+, Ura+, | Fragment of PCR ura3-41, | MTLY40, Leu+, Ura−, Δ5- |
| | Δ5-PT, Δ2-PT, Δ3-PT, | 5FOA selection | PT, Δ2-PT, Δ3-PT, Δ4- |
| | Δ4-PUT | | Pura3-41T |
| 2 | MTLY40, Leu+, Ura−, Δ5- | PHTleu2 cassette, | MTLY64, Leu−, Ura−, |
| | PT, Δ2-PT, Δ3-PT, Δ4- | hygromycin selection | Hyg+, Δ5-PT, Δ2-PT, Δ3- |
| | Pura3-41T | | PT, Δ4-Pura3-41T, |
| | | | Leu2::Hyg |
| 3 | MTLY64, Leu−, Ura−, | pRRQ2 vector, Leu+ | MTLY66, Leu−, Ura−, Δ5- |
| | Hyg+, Δ5-PT, Δ2-PT, Δ3- | selection, checking Hyg−, | PT, Δ2-PT, Δ3-PT, Δ4- |
| | PT, Δ4-Pura3-41T, | loss of plasmid pRRQ2 | Pura3-41T, Δleu2 |
| | Leu2::Hyg | on YPD, isolation of Leu− | |
| 4 | MTLY66, Leu−, Ura−, Δ5- | Expression cassette | MTLY74, Leu+, Ura−, Δ5- |
| | PT, Δ2-PT, Δ3-PT, Δ4- | pPOX2-CPR of JM21- | PT, Δ2-PT, Δ3-PT, Δ4- |
| | Pura3-41T, Δleu2 | CPR | Pura3-41T, CPR |

3. Obtaining Strains MTLY79, MTLY80 and MTLY81 from the Common Mutant MTLY74

3a. MTLY79 expressing the NADPH-cytochrome reductase and the cytochrome 10 P450 monooxygenase ALK1 under the control of promoter pPOX2 inducible by the fatty acids, fatty acid esters or natural oils.

From mutant MTLY74, we construct a mutant Yarrowia lipolytica strain MTLY79 that overexpresses NADPH-cytochrome reductase and cytochrome P450 monooxygenase ALK1 under the bioconversion conditions, under the control of the strong promoter pPOX2 induced by the bioconversion substrates of fatty acid, fatty acid ester or natural oil type.

We introduce the ALK1 gene coding for cytochrome P450 monooxygenase in a vector containing the URA3 selection gene, JMP61 for example, under the control of promoter pPOX2 inducible by the fatty acids, fatty acid esters or natural oils. The marker-promoter-gene cassette (URA3-pPOX2-AKL1) is introduced by conversion

The diagrammatic representation of the construction of this mutant is summed up in Table 3 hereunder.

TABLE 3
Conversion stage required for the production of mutant MTLY79
overexpressed for ALK1 and CPR from MTLY74

| | Conversion operation | | |
|---|---|---|---|
| Stage | Mutant to be converted | Conversion with | Converted mutant |
| 1 | MTLY74, Leu+, Ura−, | JMP61-ALK1 | MTLY79, Leu+, Ura+, |
| | Δ5-PT, Δ2-PT, Δ3- | | Δ5-PT, Δ2-PT, Δ3-PT, |
| | PT, Δ4-Pura3-41T, | | Δ4-Pura3-41T, CPR, |
| | CPR | | ALK1 |

3b. MTLY80 expressing the NADPH-cytochrome reductase and the cytochrome P450 monooxygenase ALK2 under the control of promoter pPOX2 inducible by the fatty acids, fatty acid esters or natural oils.

From mutant MTLY74, we construct a mutant Yarrowia lipolytica strain MTLY80 that overexpresses the genes coding for NADPH-cytochrome reductase (CPR) and cytochrome P450 monooxygenase (ALK2) under the bioconversion conditions, under the control of the strong promoter pPOX2 induced by the bioconversion substrates of fatty acid, fatty acid ester or natural oil type.

We introduce the ALK2 gene coding for cytochrome P450 monooxygenase in a vector containing the URA3 selection gene, JMP61 for example, under the control of promoter pPOX2 inducible by the fatty acids, fatty acid esters or natural oils. The marker-promoter-gene cassette URA3-pPOX2-AKL2) is introduced by conversion.

The diagrammatic representation of the construction of this mutant is summed up in Table 4 hereunder.

TABLE 4
Conversion stage required for the production of mutant MTLY80
overexpressed for ALK2 and CPR from MTLY74

| | Conversion operation | | |
|---|---|---|---|
| Stage | Mutant to be converted | Conversion with | Converted mutant |
| 1 | MTLY74, Leu+, Ura−, | JMP61-ALK2 | MTLY80, Leu+, Ura+, |
| | Δ5-PT, Δ2-PT, Δ3- | | Δ5-PT, Δ2-PT, Δ3-PT, |
| | PT, Δ4-Pura3-41T, | | Δ4-Pura3-41T, CPR, |
| | CPR | | ALK2 |

3c. MTLY81 expressing the NADPH-cytochrome reductase without the genes of cytochrome P450 monooxygenase (ALK1 or ALK2) under the control of promoter pPOX2 inducible by the fatty acids, fatty acid esters or natural oils.

From mutant MTLY74, we construct a mutant Yarrowia lipolytica strain MTLY81 that overexpresses the gene coding for NADPH-cytochrome reductase (CPR) under the control of the strong promoter pPOX2 induced by the bioconversion substrates of fatty acid, fatty acid ester or natural oil type.

Mutant MTLY74 has been made prototrophic by transformation with the plasmid JMP61 carrying marker URA3.

The diagrammatic representation of the construction of this mutant is summed up in Table 5 hereunder.

TABLE 5
Conversion stage required for the production of mutant MTLY81
overexpressed for CPR from MTLY74

| | Conversion operations | | |
|---|---|---|---|
| | | Conversion | |
| Stage | Mutant to be converted | with | Converted mutant |
| 1 | MTLY74, Leu+, Ura−, Δ5- | JMP61 | MTLY81, Leu+, Ura+, |
| | PT, Δ2-PT, Δ3-PT, Δ4- | | Δ5-PT, Δ2-PT, Δ3-PT, |
| | Pura3-41T, CPR | | Δ4-Pura3-41T, CPR |

4. Obtaining the Mutant Strain FT120

This strain can be obtained by following the same procedure as for the construction of strain MTLY37 or strain MTLY66:

1) constructing a disruption cassette by PCR (Polymerase Chain Reaction) or by cloning, using a counter-selectable marker, for example marker URA3 (with which one can select for the Ura+ phenotype or for the Ura− phenotype), or using a marker with, on either side, a repeated sequence (allowing the recombination that is selected) or a lox sequence that is recognized by the recombinase Cre,

2) selecting the strains with the deleted gene of interest (conversion and selection of the transformers; advantageously Ura+ if the marker is URA3) and checking the gene disruption,

3) selecting the strains with the deleted marker (conversion and selection of the transformers); advantageously 5FOA^{R} if the marker is URA3 or advantageously a plasmid expressing the recombinase if the marker exhibits the Iox sequence, and checking the gene disruption.

A particular method for obtaining the mutant strain FT20 is described hereafter.

From mutant MTLY66, we construct strain FT120 Lea− Ura−, Δpox1-6.

4a. From mutant MTLY66 Δpox2-5, we construct a mutant Yarrowia lipolytica strain MTLY95 Δpox1-6 by insertion of deletion of the POX1 and POX6 genes and deletion of the marker according to the method described above.

4b. From mutant MTLY95, we construct a mutant Yarrowia lipolytica strain FT101 Leu+ Ura− that overexpresses the gene coding for NADPH-cytochrome reductase (CPR) under the bioconversion conditions, by expressing it under the control of the strong promoter pPOX2, induced by the bioconversion substrates of fatty acid, fatty acid ester or natural oil type.

We have introduced the gene coding for NADPH-cytochrome reductase in a vector containing the excisable LEU2 selection gene, JMP21-LEU2ex for example, under the control of promoter pPOX2 inducible by the fatty acids, fatty acid esters or natural oils. The marker-promoter-gene cassette (LEU2ex-pPOX2-CPR) is introduced by transformation. The FT120 strain is obtained after excision of marker LEU2ex by conversion with the pUB4-CRE plasmid, Hyg+ selection, plasmid loss on YPD and finally isolation of a Leu−clone.

The diagrammatic representation of the construction of this mutant is summed up in Table 6 hereafter.

TABLE 6
Conversion stages required for the production of mutant FT120 from MTLY66

| | Conversion operations | | |
|---|---|---|---|
| Stage | Mutant to be converted | Conversion cassette | Converted mutant |
| 1 | MTLY66, Leu−, Ura−, | POX1-PHT | MTLY82, Leu−, Ura−, |
| | Hyg−, Δ5-PT, Δ2-PT, Δ3- | | Hyg+, Δ5-PT, Δ2-PT, Δ3- |
| | PT, Δ4-Pura3-41T | | PT, Δ4-Pura3-41T, Δ1- |
| | | | PHT |
| 2 | MTLY82, Leu−, Ura−, | pRRQ2 vector, Leu+ | MTLY85 Leu−, Ura−, Hyg−, |
| | Hyg+, Δ5-PT, Δ2-PT, Δ3- | selection, checking Hyg−, | Δ5-PT, Δ2-PT, Δ3-PT, |
| | PT, Δ4-Pura3-41T, Δ1- | loss of plasmid pRRQ2 | Δ4-Pura3-41T, Δ1-PT |
| | PHT | on YPD, isolation of Leu− | |
| 3 | MTLY85 Leu−, Ura−, | POX6-PHT | MTLY92 Leu−, Ura−, |
| | Hyg−, Δ5-PT, Δ2-PT, Δ3- | | Hyg+, Δ5-PT, Δ2-PT, Δ3- |
| | PT, Δ4-Pura3-41T, Δ1- | | PT, Δ4-Pura3-41T, Δ1- |
| | PT | | PT, Δ6-PHT |
| 4 | MTLY92 Leu−, Ura−, | pRRQ2 vector, Leu+ | MTLY95 Leu−, Ura−, Hyg−, |
| | Hyg+, Δ5-PT, Δ2-PT, Δ3- | selection, checking Hyg−, | Δ5-PT, Δ2-PT, Δ3-PT, |
| | PT, Δ4-Pura3-41T, Δ1- | loss of plasmid pRRQ2 | Δ4-Pura3-41T, Δ1-PT, |
| | PT, Δ6-PHT | on YPD, isolation of Leu− | Δ6-PT |
| 5 | MTLY95 Leu−, Ura−, | Expression cassette | FT101, Leu+, Ura−, Hyg−, |
| | Hyg−, Δ5-PT, Δ2-PT, Δ3- | pPOX2-CPR of JM21- | Δ5-PT, Δ2-PT, Δ3-PT, |
| | PT, Δ4-Pura3-41T, Δ1- | LEU2ex-CPR | Δ4-Pura3-41T, Δ1-PT, |
| | PT, Δ6-PT | | Δ6-PT, CPR-LEU2ex |
| 6 | FT101, Leu+, Ura−, Hyg−, | pUB4-CRE vector, Hyg+ | FT120, Leu−, Ura−, Hyg−, |
| | Δ5-PT, Δ2-PT, Δ3-PT, | selection, checking Leu−, | Δ5-PT, Δ2-PT, Δ3-PT, |
| | Δ4-Pura3-41T, Δ1-PT, | loss of plasmid pUB4- | Δ4-Pura3-41T, Δ1-PT, |
| | Δ6-PT, CPR-LEU2ex | CRE on YPD, isolation of | Δ6-PT, CPR |
| | | Hyg− | |

5. Obtaining the Mutant Strain FT130

This strain can be obtained by following the same procedure as for the construction of strain FT 120:

1) constructing a disruption cassette by PCR (Polymerase Chain Reaction) or by cloning, using a counter-selectable marker, for example marker URA3 (with which one can select for the Ura+ phenotype or for the Ura− phenotype), or using a marker with, on either side, a repeated sequence (allowing the recombination that is selected) or a lox sequence that is recognized by the recombinase Cre,

2) selecting the strains with the deleted gene of interest (conversion and selection of the transformers; advantageously Ura+ if the marker is URA3) and checking the gene disruption,

3) selecting the strains with the deleted marker (conversion and selection of the transformers); advantageously 5FOA^{R} if the marker is URA3 or advantageously a plasmid expressing the recombinase Cre if the marker exhibits the lox sequence, and checking the gene disruption.

A particular method for obtaining the mutant strain FT130 is described hereafter.

From mutant FT120, we construct strain FT130 Leu− Ura−, Δpox 1-6, Δdga1.

6. From mutant FT120 Leu− Ura−, Δpox1-6, pPOX2-CPR, we construct a mutant Yarrowia lipolytica strain FT130 Leu− Ura+ Δpox1-6, pPOX2-CPR, Δdga1 by insertion of deletion of the DGA1 gene coding for acyl-CoA diacylglycerol acyltransferase.

TABLE 7
Conversion stage required for the production of mutant FT130 from
FT120

| | Conversion operations | | |
|---|---|---|---|
| | | Conversion | |
| Stage | Mutant to be converted | cassette | Converted mutant |
| 1 | FT120, Leu−, Ura−, | DGA1-PUT | FT130, Leu−, Ura+, Hyg−, |
| | Hyg−, Δ5-PT, Δ2-PT, | | Δ5-PT, Δ2-PT, Δ3-PT, |
| | Δ3-PT, Δ4-Pura3-41T, | | Δ4-Pura3-41T, Δ1-PT, |
| | Δ1-PT,Δ6-PT, CPR | | Δ6-PT, CPR, Δdga1-PUT |

Strains MTLY66, MTLY81, FT120 and FT130 are registered at the Collection Nationale de Cultures de Microorganismes under the respective registration numbers CNCM I-3319, CNCM I-3320, CNCM I-3527 and CNCM I-3528.

The following examples illustrate the invention without limiting the scope thereof.

### EXAMPLES

In these examples, we have tested the influence of the culture conditions and of the medium composition on the production of diacids. We have thus observed with mutant MTLY37 that the use of peptone greatly favours the production of diacids, notably in relation to bacto-tryptone (Examples 1 and 2).

We have also tested, under the same conditions, mutants MTLY79, MTLY80 and MTLY81. We have thus observed that NADPH-cytochrome reductase catalyzes a limiting stage in the production of diacids. In fact, overexpression of this enzyme alone allows to significantly increase the production and the productivity of diacids (Examples 4 to 6).

We have tested, under identical conditions, the mutant strains PT120 and FT130 (Examples 7 and 8). Deletion of the POX1 and POX6 genes allows to decrease the diacids degradation for FT120. Deletion of an additional DGA1 gene coding for acyl-CoA diacylglycerol acyltransferase leads to a decrease in the accumulation of bioconversion substrate in form of lipid bodies within the Yarrowia lipolytica cell. Thus, the major part of the diacids obtained in these examples consists of diacids with 18 carbon atoms like the bioconversion substrate used, essentially consisting of fatty acids with 18 carbon atoms.

### Example 1

### Method of Producing Dicarboxylic Acids from Oleic Sunflower Oil with Mutant MTLY37

A preculture of mutant MTLY37, kept in a gelosed medium of composition: yeast extract 10 g·l⁻¹, peptone 10 g·l⁻¹, glucose 10 g·l⁻¹, Agar 20 g·l⁻¹, is carried out by means of a seeding that provides an initial absorbance of the preculture medium close to 0.30. The preculture is performed under orbital stirring (200 rpm) for 24 h at 30° C. in a 500-ml flanged flask containing 25 ml of medium (10 g·l⁻¹ yeast extract, 10 g·l⁻¹ peptone, 20 g·l⁻¹ glucose).

The medium used for culture is made up of deionized water, 10 g·l⁻¹ yeast extract, 20 g·l⁻¹ tryptone, 40 g·l⁻¹ glucose and 30 g·l⁻¹ oleic sunflower oil.

Seeding of the fermenter is achieved with all of the preculture flask.

Culture is carried out at 30° C. in a 4-l fermenter with 2 l medium at an aeration rate of 0.5 vvm and a stirring speed of 800 rpm provided by a double-acting centripetal turbine.

After 17 hours culture, as soon as the glucose of the medium has run out, 60 ml oleic sunflower oil, essentially consisting of fatty acids with 18 carbon atoms, are added into the reactor that is subjected to a continuous glycerol supply at a rate of 1 ml·h⁻¹. The pH value of the culture is then maintained at a constant value of 8 by adjusted addition of 4M soda. Fermentation lasts for 130 h.

At the end of the culture procedure, the cellular biomass is removed by centrifugation. The supernatent is then acidized up to a pH value of 2.5 by adding 6M HCl and the insoluble dicarboxylic acids are collected by centrifugation of the acidized wort, then dried.

The dicarboxylic acid composition of the mixture is determined by gas chromatography in a column DB1 after conversion of the dicarboxylic acids to diesters according to the method described by Uchio et al.: Microbial Production of Long-chain Dicarboxylic Acids from n-Alkanes. Part II. Production by Candida cloacae Mutant Unable to Assimilate Dicarboxylic Acid. Agr Biol. Chem. 36, No. 3, 1972, 426-433. The temperature of the chromatograph oven is programmed from 150° C. to 280° C. at a rate of 8° C./min.

The results show a maximum diacid production in the i of 5.9 g·l⁻¹ after 130 h.

### Example 2

Example 1 is repeated by replacing, in the culture medium, the tryptone by peptone at the same concentration. After 130 h culture, 9.9 g·l⁻¹ dicarboxylic acids are obtained, i.e. a production increase of about 68% in relation to Example 1.

### Example 3

### Production of Dicarboxylic Acids by Mutant MTLY37 with Continuous Oleic Sunflower Oil Supply

Example 2 is repeated, the oleic sunflower oil being removed from the culture medium and replaced by continuous injection of this oil at a sublimiting flow of 1 ml in the reactor.

Under such conditions, 14.7 g·l⁻¹ dicarboxylic acids are produced in the culture medium after 130 h.

### Example 4

### Production of Dicarboxylic Acids from Oleic Sunflower Oil with Mutant MTLY79 Overexpressed for CPR and ALK1

Example 3 is repeated by replacing mutant MTLY37 by mutant MTLY79 overexpressing the CPR and ALK1 genes. After 130 h culture, 16 g·l⁻¹ dicarboxylic acids are obtained.

### Example 5

### Production of Dicarboxylic Acids from Oleic Sunflower Oil with Mutant MTLY80 Overexpressed for CPR and ALK2

Example 3 is repeated by replacing mutant MTLY37 by mutant MTLY80 overexpressing the CPR and ALK2 genes. After 130 h culture, 16 g·l⁻¹ dicarboxylic acids are obtained.

### Example 6

Example 3 is repeated by replacing mutant MTLY37 by mutant MTLY81 overexpressing only the CPR gene. After 130 h culture, 16 g·l⁻¹ dicarboxylic acids are obtained.

### Example 7

### Production of Dicarboxylic Acids from Oleic Sunflower Oil with Mutant FT120 Deleted for the Six POX (Δpox1-6) Genes and Overexpressing the CPR Gene

Example 3 is repeated by replacing mutant MTLY37 by mutant FT120 deleted of the six POX genes and overexpressing only the CPR gene. After 130 h culture, 18 g·l⁻¹ dicarboxylic acids are obtained.

### Example 8

### Production of Dicarboxylic Acids from Oleic Sunflower Oil with Mutant FT130 Deleted for the Six POX (Δpox1-6) Genes and Deleted for the DGA1 Gene (Δdga1) and Overexpressing the CPR Gene

Example 3 is repeated by replacing mutant MTLY37 by mutant FT130 deleted of the six POX genes and of the DGA1 gene and overexpressing the CPR gene. After 130 h culture, 23 g·l⁻¹ dicarboxylic acids are obtained.

1. A method of producing dicarboxylic acids, comprising:
(a) a growth stage wherein a mutant strain of Yarrowia lipolytica disrupted at least for the POX2, POX3, POX4 and POX5 genes (coding for acyl-CoA oxidase) is cultured in a culture medium essentially consisting of an energetic substrate comprising at least a source of carbon and a source of nitrogen,
(b) a bioconversion stage wherein said strain is subjected to a bioconversion substrate selected from among the n-alkanes having at least 10 carbon atoms, fatty acids having at least 10 carbon atoms, alkyl esters having 1 to 4 carbon atoms of these fatty acids and natural oils, in the presence of an energetic substrate, and
(c) a stage of recovering the dicarboxylic acid formed.

2. A method as claimed in claim 1, characterized in that the mutant used is MTLY37.

3. A method as claimed in claim 1, characterized in that the mutant used is MTLY79 overexpressing the CPR and ALK1 genes.

4. A method as claimed in claim 1, characterized in that the mutant used is MTLY80 overexpressing the CPR and ALK2 genes.

5. A method as claimed in claim 1, characterized in that the mutant used is MTLY81 overexpressing the CPR gene.

6. A method as claimed in claim 1, characterized in that the mutant used is FT120 overexpressing the CPR gene.

7. A method as claimed in claim 1, characterized in that the mutant used is FT130 overexpressing the CPR gene.

8. A method as claimed in claim 1, characterized in that said bioconversion substrate consists of a mixture of methyl esters or of a mixture of ethyl esters.

9. A method as claimed in claim 1, characterized in that said bioconversion substrate consists of an oleic sunflower oil.

10. A method as claimed in claim 1, characterized in that, in the bioconversion stage, the culture medium comprises peptone.

11. A method as claimed in claim 1, characterized in that, in the bioconversion stage, the culture medium comprises a supply of secondary energetic substrate consisting of at least one polyhydroxyl compound.

12. A method as claimed in claim 9, characterized in that said polyhydroxyl compound is glycerol or a sugar.

13. A method as claimed in claim 1, characterized in that, in stage (c), the dicarboxylic acid is recovered by precipitation in form of calcium salt.

14. A method of obtaining a mutant Yarrowia lipolytica auxotrophic strain MTLY66, Leu− Ura−, from the prototrophic mutant MTLY37, usable for transformation with, as the selection markers, the LEU2 and URA3 genes, characterized in that the conversion operations of stages 1 to 3 of the table hereafter are carried out:

| | Conversion operations | | |
|---|---|---|---|
| Stage | Mutant to be converted | Conversion with | Converted mutant |
| 1 | MTLY37, Leu+, Ura+, | Fragment of PCR ura3- | MTLY40, Leu+, Ura−, Δ5- |
| | Δ5-PT, Δ2-PT, Δ3-PT, | 41, 5FOA selection | PT, Δ2-PT, Δ3-PT, |
| | Δ4-PUT | | Δ4-Pura3-41T |
| 2 | MTLY40, Leu+, Ura−, Δ5- | PHTleu2 cassette, | MTLY64, Leu−, Ura−, |
| | PT, Δ2-PT, Δ3-PT, | hygromycin selection | Hyg+, Δ5-PT, Δ2-PT, Δ3- |
| | Δ4-Pura3-41T | | PT, Δ4-Pura3-41T, |
| | | | Leu2::Hyg |
| 3 | MTLY64, Leu−, Ura−, | pRRQ2 vector, Leu+ | MTLY66, Leu−, Ura−, Δ5- |
| | Hyg+, Δ5-PT, Δ2-PT, Δ3- | selection, checking Hyg−, | PT, Δ2-PT, Δ3-PT, |
| | PT, Δ4-Pura3-41T, | loss of plasmid pRRQ2 | Δ4-Pura3-41T, Δleu2 |
| | Leu2::Hyg | on YPD, isolation of Leu− | |

15. A method of obtaining, from mutant MTLY66, a mutant Yarrowia lipolytica strain MTLY74 Leu+ Ura− that overexpresses the CPR gene coding for NADPH-cytochrome reductase under the bioconversion conditions, by conversion of the JMP21-CPR vector containing selection marker LEU2 and the expression cassette with the CPR gene under the control of promoter pPOX2 inducible by the fatty acids, fatty acid esters or natural oils.

16. A method of obtaining, from mutant Yarrowia lipolytica strain MTLY74, a mutant Yarrowia lipolytica strain MTLY79 that overexpresses the genes coding for NADPH-cytochrome reductase and for cytochrome P450 monooxygenase under the bioconversion conditions, by conversion of the JMP21-ALK1 vector containing selection marker URA3 and the expression cassette with the ALK1 gene under the control of promoter pPOX2 inducible by the fatty acids, fatty acid esters or natural oils.

17. A method of obtaining, from mutant Yarrowia lipolytica strain MTLY74, a mutant Yarrowia lipolytica strain MTLY80 that overexpresses the genes coding for NADPH-cytochrome reductase and for cytochrome P450 monooxygenase ALK2 under the bioconversion conditions, by conversion of the JMP61-ALK2 vector containing selection marker URA3 and the expression cassette with the ALK2 gene under the control of promoter pPOX2 inducible by the fatty acids, fatty acid esters or natural oils.

18. A method of obtaining a mutant Yarrowia lipolytica strain MTLY81 from mutant strain MTLY74, that overexpresses the CPR gene coding for NADPH-cytochrome reductase under the bioconversion conditions, by expressing it under the control of promoter pPOX2 induced by the bioconversion substrates, of fatty acid, fatty acid ester or natural oil type and that is prototrophic, by making mutant MTLY 74 prototrophic by transformation with the JMP61 plasmid carrying marker URA3.

19. A method of obtaining, from mutant Yarrowia lipolytica strain MTLY66, a mutant Yarrowia lipolytica strain FT120 Leu− Ura−, characterized in that the conversion operations of stages 1 to 4 of the table hereafter are carried out:

| | Conversion operations | | |
|---|---|---|---|
| Stage | Mutant to be converted | Conversion cassette | Converted mutant |
| 1 | MTLY66, Leu−, Ura−, | POX1-PHT | MTLY82, Leu−, Ura−, |
| | Hyg−, Δ5-PT, Δ2-PT, Δ3- | | Hyg+, Δ5-PT, Δ2-PT, Δ3- |
| | PT, Δ4-Pura3-41T | | PT, Δ4-Pura3-41T, Δ1- |
| | | | PHT |
| 2 | MTLY82, Leu−, Ura−, | pRRQ2 vector, Leu+ | MTLY85 Leu−, Ura−, Hyg−, |
| | Hyg+, Δ5-PT, Δ2-PT, Δ3- | selection, checking Hyg−, | Δ5-PT, Δ2-PT, Δ3-PT, |
| | PT, Δ4-Pura3-41T, Δ1- | loss of plasmid pRRQ2 | Δ4-Pura3-41T, Δ1-PT |
| | PHT | on YPD, isolation of Leu− | |
| 3 | MTLY85 Leu−, Ura−, Hyg−, | POX6-PHT | MTLY92 Leu−, Ura−, |
| | Δ5-PT, Δ2-PT, Δ3-PT, | | Hyg+, Δ5-PT, Δ2-PT, Δ3- |
| | Δ4-Pura3-41T, Δ1-PT | | PT, Δ4-Pura3-41T, Δ1- |
| | | | PT, Δ6-PHT |
| 4 | MTLY92 Leu−, Ura−, | pRRQ2 vector, Leu+ | MTLY95 Leu−, Ura−, Hyg−, |
| | Hyg+, Δ5-PT, Δ2-PT, Δ3- | selection, checking Hyg−, | Δ5-PT, Δ2-PT, Δ3-PT, |
| | PT, Δ4-Pura3-41T, Δ1- | loss of plasmid pRRQ2 | Δ4-Pura3-41T, Δ1-PT, |
| | PT, Δ6-PHT | on YPD, isolation of Leu− | Δ6-PT |
| 5 | MTLY95 Leu−, Ura−, Hyg−, | Expression cassette | FT101, Leu+, Ura−, Hyg−, |
| | Δ5-PT, Δ2-PT, Δ3-PT, | pPOX2-CPR of JM21- | Δ5-PT, Δ2-PT, Δ3-PT, |
| | Δ4-Pura3-41T, Δ1-PT, | LEU2ex-CPR | Δ4-Pura3-41T, Δ1-PT, |
| | Δ6-PT | | Δ6-PT, CPR-LEU2ex |
| 6 | FT101, Leu+, Ura−, Hyg−, | pUB4-CRE vector, Hyg+ | FT120, Leu−, Ura−, Hyg−, |
| | Δ5-PT, Δ2-PT, Δ3-PT, | selection, checking Leu−, | Δ5-PT, Δ2-PT, Δ3-PT, |
| | Δ4-Pura3-41T, Δ1-PT, | loss of plasmid pUB4- | Δ4-Pura3-41T, Δ1-PT, |
| | Δ6-PT, CPR-LEU2ex | CRE on YPD, isolation of | Δ6-PT, CPR |
| | | Hyg− | |

and in that construction of the mutant strain FT101 Leu+ Ura− is performed from Yarrowia lipolytica strain MTLY95, that overexpresses the gene coding (CPR) for NADPH-cytochrome reductase under the bioconversion conditions, by conversion of the JMP21-LEU2ex-CPR vector containing the excisable selection marker LEU2 and the CPR gene under the control of promoter pPOX2 inducible by the fatty acids, fatty acid esters or natural oils, strain FT120 being obtained after excision of marker LEU2ex by conversion with the pUB4-CRE plasmid, Hyg+ selection, loss of plasmid on YPD and finally isolation of a clone Leu−.

20. A method of obtaining from mutant Yarrowia lipolytica strain FT120, a mutant Yarrowia lipolytica strain FT130, characterized in that the following conversion stage is carried out:

| | Conversion operations | | |
|---|---|---|---|
| | | Conversion | |
| Stage | Mutant to be converted | cassette | Converted mutant |
| 1 | FT120, Leu−, Ura−, | DGA1-PUT | FT130, Leu−, Ura+, Hyg−, |
| | Hyg−, Δ5-PT, Δ2-PT, | | Δ5-PT, Δ2-PT, Δ3-PT, |
| | Δ3-PT, Δ4-Pura3-41T, | | Δ4-Pura3-41T, Δ1-PT, |
| | Δ1-PT, Δ6-PT, CPR | | Δ6-PT, CPR, Δdga1-PUT |

21. A new mutant Yarrowia lipolytica strain MTLY66.

22. A new mutant Yarrowia lipolytica strain MTLY81.

23. A new mutant Yarrowia lipolytica strain FT120.

24. A new mutant Yarrowia lipolytica strain FT130.
